(19) United States
(12) Patent Application Publication
(10) Pub. No.: US 2015/0133543 A1
(43) Pub. Date: May 14, 2015

(54) TOPICAL GEL COMPOSITION COMPRISING AN INGENOL DERIVATIVE AND A SOLVENT MIXTURE

(71) Applicant: LEO Laboratories Limited, Dublin (IE)

(72) Inventors: Per-Ola Arvidsson, Ballerup (DK); Edit Farkas, Copenhagen NV (DK); Karsten Petersson, Ballerup (DK); Chinar S. Saeed, Gentofte (DK)

(73) Assignee: LEO Laboratories Limited, Dublin (IE)

(21) Appl. No.: 14/406,509

(22) PCT Filed: Jun. 7, 2013
(86) PCT No.: PCT/EP2013/061825

§ 371 Date: Dec. 8, 2014

Related U.S. Application Data
(60) Provisional application No. 61/657,258, filed on Jun. 8, 2012.

(30) Foreign Application Priority Data
Dec. 12, 2012 (GB) 1222402.8

(51) Int. Cl. A61K 47/44; A61K 31/22; A61K 47/06; A61K 47/14; A61K 47/12; A61K 9/00; A61K 47/10

(52) U.S. Cl. CPC A61K 47/44; A61K 9/0014; A61K 31/22; A61K 47/10; A61K 47/14; A61K 47/12; A61K 47/06
USPC 514/511

(57) ABSTRACT

A substantially anhydrous pharmaceutical gel composition for cutaneous application comprising an ingenol derivative in dissolved or solubilized form, a solvent mixture of
(a) a hydrophilic non-ionic surfactant;
(b) a lipophilic non-ionic co-surfactant;
(c) an oil;
the composition further comprising an acidic compound, a co-solvent, a viscosity-increasing agent and
a pharmaceutically acceptable, substantially anhydrous, non-solvent lipid carrier.

### FIELD OF INVENTION

The present invention relates to a topical pharmaceutical gel formulation comprising a pharmacologically active agent and a solvent mixture comprising an oil for the active agent and two different surfactants.

### BACKGROUND OF THE INVENTION

The invention provides a pharmaceutical formulation suitable for topical application of the compound ingenol-3-angelate (2-methyl-2(Z)-butenoic acid (1aR,2S,5R,5aS,6S,8aS,9R,10aR)-5,5a-dihydroxy-4-(hydroxymethyl)-1,1,7,9-tetramethyl-11-oxo-1a,2,5,5a,6,9,10,10a-octahydro-1H-2,8a-methanocyclopenta[a]cyclopropa[e]cyclodecen-6-yl ester; PEP005).

Ingenol-3-angelate (PEP005) is a protein kinase C activator in phase III clinical development for the treatment of actinic keratosis. The drug candidate is also in phase II trials for non-melanoma skin cancer [Ogbourne, S. M.; Anti-cancer Drugs, (2007), 18, 357-62].

The compound ingenol-3-angelate (PEP005) [Sayed, M. D. et.al.; Experienta, (1980), 36, 1206-1207] can be isolated from various Euphorbia species, and particularly from Euphorbia peplus [Hohmann, J. et. al; Planta Med., (2000), 66, 291-294] and Euphorbia drummondii by extraction followed by chromatography as described in U.S. Pat. No. 7,449,492. Pharmaceutical formulation of the compound has been described in WO200768963.

Angelic acid and angelic acid esters, as present in ingenol-3-angelate, are prone to isomerisation of the double bond to form the tiglate ester, particularly at basic pH [Beeby, P., Tetrahedron Lett. (1977), 38, 3379-3382, Hoskins, W. M., J. Chem. Soc. Perkin Trans. 1, (1977), 538-544, Bohlmann, F. et. al., Chem. Ber. (1970), 103, 561-563]. As a consequence only carefully optimised conditions for ester formation can be applied in the synthetic preparation of ingenol-3-angelate.

Furthermore, ingenol-3-acylates are known to be unstable as they rearrange to afford the ingenol-5-acylates and ingenol-20-acylates [Sorg, B. et. al, Z. Naturforsch., (1982), 37B, 748-756].

WO 2007/068963 discloses a gel formulation for the treatment of skin cancer in which ingenol angelate is dissolved in a solvent such as benzyl alcohol or isopropanol, the formulation further comprising an acidifying agent such that the pH of the formulation is no greater than 4.5. The formulation is stable at a temperature of 2-8° C. One object of the invention is therefore to provide a composition of the ingenol derivative which is stable at room temperature for the shelf-life of the composition.

Another object of the invention is to provide a composition exhibiting favourable penetration characteristics and biological activity.

A further object of the invention is to provide a composition with reduced skin irritation, favourable cosmetic properties and improved patient compliance

### SUMMARY OF THE INVENTION

Human skin, in particular the outer layer, the stratum corneum, provides an effective barrier against penetration of microbial pathogens and toxic chemicals. While this property of skin is generally beneficial, it complicates the dermal administration of pharmaceuticals in that a large quantity, if not most, of the active ingredient applied on the skin of a patient suffering from a dermal disease may not penetrate into the viable layers of the skin where it exerts its activity. To ensure an adequate penetration of the active ingredient to the dermis and epidermis, it is generally preferred to include the active ingredient in a dissolved state, typically in the presence of a solvent in the form of an alcohol, e.g. benzyl alcohol or isopropanol, or a diol, e.g. propylene glycol. Propylene glycol is a well-known penetration enhancer, i.e. a substance which is capable of penetrating the stratum corneum and “draw” low-molecular components such as therapeutically active components in the vehicle into the epidermis. Alcohols such as isopropanol and diols may in themselves give rise to significant skin irritation if used in high quantities (e.g. 30% by weight as in the compositions disclosed in WO 2007/068963), and they are also capable of “drawing” low-molecular and potentially irritative components of the vehicle into the epidermis, leading to an overall irritative effect of conventional vehicles including an alcohol or a diol.

In the research leading to the present invention, it was surprisingly found that certain three-component surfactant-oil mixtures of a type which self-emulsifies in the presence of an excess of water to form microemulsions are suitable for inclusion in topical compositions for application on skin. The mixtures also exhibit a good solubilization capacity in dissolving sparingly water-soluble active ingredients such as ingenol derivatives. The compositions are easily spreadable, and therefore likely to improve patient compliance, and exhibit an adequate physical and chemical stability.

Accordingly, in one aspect the present invention relates to a substantially anhydrous pharmaceutical gel composition for cutaneous application comprising an ingenol derivative in dissolved or solubilized form, a solvent mixture of

(a) a hydrophilic non-ionic surfactant;

(b) a lipophilic non-ionic co-surfactant;

(c) an oil;

the composition further comprising an acidic compound, a co-solvent, a viscosity-increasing agent and

a pharmaceutically acceptable, substantially anhydrous, non-solvent lipid carrier.

The present composition has been found to result in an improved chemical stability of the ingenol derivative included therein (cf. Example 2 below) permitting the composition to be stored at room temperature (about 25° C.). The improved storage stability may partly be ascribed to the absence of water from the composition and partly to the presence of an acidic compound which may provide an acidic environment in the composition and neutralize alkaline components such as alkaline impurities that may be detrimental to the stability of the ingenol derivative.

Furthermore, it is well known that a high concentration of surfactant in a topical composition for dermatological use may result in increased skin irritation. By providing a composition containing a lower amount of surfactant and by including lipid components that are compatible with the structure of the skin, the risk of skin irritation resulting from the use of irritative excipients may be reduced.

The present composition may in addition exhibit reduced skin irritation due to the absence or reduced amounts of irritative excipients such as isopropanol or inclusion of lipid components that are compatible with the structure of the skin.

Solvent mixtures of the type included in the present compositions (i.e. self-microemulsifying in the presence of an excess of water) have been described in the literature. Thus, U.S. Pat. No. 5,645,856 discloses a pharmaceutical composition comprising a hydrophobic drug, a digestible oil, a hydrophilic surfactant and a lipophilic surfactant. The composition is intended to increase the solubility of the hydrophobic drug on oral administration in that the oil-surfactant mixture self-emulsifies in gastric fluid resulting in the formation of a microemulsion claimed to result in faster and more complete absorption of the drug. There is no indication that that the solvent mixtures disclosed in U.S. Pat. No. 5,645,856 could be incorporated in compositions intended for dermal application.

U.S. Pat. No. 5,948,825 discloses a water-in-oil microemulsion comprising an oil phase, an aqueous phase and a combination of hydrophilic and lipophilic surfactants, the dispersed oil droplets of the microemulsion having a particle size of 0.4-100 nm. Said microemulsions are intended for systemic delivery of pharmaceutically active proteins dissolved in an aqueous phase, or to improve the bioavailability of low molecular weight drugs. There is no indication that the microemulsions disclosed in U.S. Pat. No. 5,948,825 could be incorporated in a composition intended for dermal application.

U.S. Pat. No. 6,267,985 discloses a composition comprising a triglyceride and either two hydrophilic surfactants or one hydrophilic and one lipophilic surfactant as well as an active ingredient solubilized in the triglyceride or triglyceride-surfactant mixture. The composition forms a clear aqueous dispersion when mixed with water in a ratio of 1:100. The composition is intended for oral administration to provide improved absorption of the active ingredient in the gastrointestinal tract. There is no suggestion of mixing the composition with excipients that would make it appropriate for cutaneous application.

M. Grove et al., European Journal of Pharmaceutical Sciences 28, 2006, pp. 233-242, disclose a drug delivery system comprising a lipid, surfactant and co-surfactant as well as a vitamin D analogue (seocalcitol) as the active ingredient. On dilution with water, the system formed microemulsions with a droplet size of 30 nm. On oral administration to rats, the bioavailability of seocalcitol was not improved over a formulation in lipid alone, and the chemical stability had decreased below the acceptable limit after 3 months at 40° C./75% RH. There is no indication that the drug delivery system disclosed in Grove et al. is suitable of incorporation in a composition intended for dermal application or that it may be possible to obtain an adequate chemical stability of an ingenol derivative included in such a composition.

The composition of the invention differs from those disclosed in these publications by being intended for cutaneous application and by comprising one or more excipients that are suitable for dermal use. In particular, the substantially anhydrous lipid carrier is expected to provide an occlusive layer on the skin surface on which the composition is applied such that moisture evaporating or secreted from the skin accumulates between the skin surface and the occlusive layer. While the amount of moisture is not expected to be sufficient to cause self-emulsification of the solvent mixture to form a microemulsion, such as is disclosed in the publications mentioned above, it is presumed to result in the formation of ordered structures such as liquid crystalline, lamellar phases or micelles comprising the solubilized or dissolved active ingredient, depending on the amount of water present. It is currently believed that the presence of surfactant and co-surfactant in the composition may contribute to the penetration of the active ingredient as the surfactant(s) may modulate the cellular membrane to increase its permeability to small chemical entities such as ingenol derivatives.

In another aspect of the invention, the present composition may be used in the treatment of a dermal disease or condition.

### DETAILED DESCRIPTION OF THE INVENTION

### Definitions

In the present context, the term “non-ionic surfactant” is intended to indicate a surfactant comprising a hydrophilic and a hydrophobic portion in which the hydrophilic portion carries no charge but derives its surface activity from highly polar groups such as polyoxyethylene groups. For the present purpose, a hydrophilic surfactant is understood to be an oil-in-water surfactant with an HLB value of 9-18, and a lipophilic surfactant is understood to be a water-in-oil surfactant with an HLB value of 2-9.

The term “ingenol derivative” is intended to mean an ingenol compound isolated from a species of Euphorbia, in particular from E. peplus. Examples of ingenol derivatives that may be included in the present compositions are ingenol-3-angelate, ingenol-5-angelate, ingenol-20-angelate, 20-O-acetyl-ingenol-3-angelate and 20-deoxy-ingenol-3-angelate. Ingenol-3-angelate, also known as ingenol-3-mebutate or PEP 005, is currently in development for the treatment of actinic keratosis. Further examples of ingenol derivatives that may be included in the present composition are disclosed in copending application No. PCT/DK2011/000154, including

Ingenol 3-(5-methyl-3-phenyl-isoxazole-4-carboxylate) or

Ingenol 3-(5-methyl-3-(2-chloro-6-fluoro-phenyl)-isoxazole-4-carboxylate) or

Ingenol 3-(1S-camphanate) or

Ingenol 3-(3-phenyltriazole-4-carboxylate or

Ingenol 3-(2-phenylpyrazole-3-carboxylate or

Ingenol 3-(1-methylindazole-3-carboxylate) or

Ingenol 3-(3-ethyl-5-methyl-isoxazole-4-carboxylate) or

Ingenol 3-(3-methyl-5-methyl-isoxazole-4-carboxylate) or

Ingenol 3-(1-methylindole-3-carboxylate) or

Ingenol 3-(3-phenylthiophene-2-carboxylate) or

Ingenol 3-(5-phenylisoxazole-3-carboxylate) or

Ingenol 3-(N-ethyl-carbamate) or

Ingenol 3-(N,N-dimethyl-carbamate) or

Ingenol 3-(morpholine-4-carboxylate) or

Ingenol 3-(pyrrolidine-1-carboxylate) or

Ingenol 3-(N-methyl-N-phenyl-carbamate) or

Ingenol 3-(N,N-diethyl-carbamate) or

Ingenol 3-(piperidine-1-carboxylate) or

Ingenol 3-(N-benzyl-N-methyl-carbamate) or

Ingenol 3-(N-cyclohexyl-N-methyl-carbamate) or

Ingenol 3-(N-cyclohexyl-carbamate) or

Ingenol 3-(N-phenyl-carbamate) or

Ingenol 3-(N-(indan-1-yl)-carbamate) or

Ingenol 3-(3,3-dimethyl-piperidine-1-carboxylate) or

Ingenol 3-(N-Methyl-N-tetralin-1-yl-carbamate) or

Ingenol 3-(N-(2-cyano-1-methyl-ethyl)-N-methyl-carbamate) or

Ingenol 3-(N-methyl-N-((S)-1-phenethyl)-carbamate) or

Ingenol 3-(N-methyl-N-(cyclopropylmethyl)-carbamate) or

Ingenol 3-(isoquinoline-1-carboxylate) or

Ingenol 3-(quinoline-4-carboxylate) or

Ingenol 3-(cinnoline-4-carboxylate) or

Ingenol 3-(3-phenylimidazole-4-carboxylate) or

Ingenol 3-(5-phenyloxazole-4-carboxylate) or

Ingenol 3-(1,2-benzoxazole-3-carboxylate) or

Ingenol 3-(3-isopropyl-5-methyl-isoxazole-4-carboxylate) or

Ingenol 3-(3-(2-methoxyphenyI)-5-methylsoxazole-4-carboxylate) or

Ingenol 3-(4-bromo-2-methyl-pyrazole-3-carboxylate) or

Ingenol 3-(4-bromo-2-ethyl-pyrazole-3-carboxylate) or

Ingenol 3-(4-chloro-2-methyl-pyrazole-3-carboxylate) or

Ingenol 3-(5-bromopyrimidine-4-carboxylate) or

Ingenol 3-(3-bromopyridine-2-carboxylate) or

Ingenol 3-(5-methylthiazole-4-carboxylate) or

Ingenol 3-(4-chloro-1-methyl-pyrazole-3-carboxylate) or

Ingenol 3-(2,4-dimethylthiazole-5-carboxylate) or

Ingenol 3-(2,5-dimethyioxazole-4-carboxylate) or

Ingenol 3-(2,4-dimethyifuran-3-carboxylate) or

Ingenol 3-(3,5-diethylisoxazole-4-carboxylate) or

Ingenol 3-(N-(3-fluoro-phenyl)-N-methyl-carbamate) or

Ingenol 3-(N-(2,5-dimethylpyrazol-3-yl)-N-methyl-carbamate) or

Ingenol 3-(1H-indole-7-carboxylate) or

Ingenol 3-(2-tent-butyl-5-methyl-pyrazole-3-carboxylate) or

Ingenol 3-(5-tert-butyl-2-methyl-pyrazole-3-carboxylate) or

Ingenol 3-(6-methylimidazo[2,1-b]thiazole-5-carboxylate) or

Ingenol 3-(2-methylimidazo[1,2-a]pyridine-3-carboxylate) or

Ingenol 3-(2,4,5-trimethylfuran-3-carboxylate) or

Ingenol 3-(3-methylthiophene-2-carboxylate) or

Ingenol 3-(2-methyl-4-(1-piperidyl)pyrazole-3-carboxylate) or

Ingenol 3-(2-chloro-5-isopropyl-thiazole-4-carboxylate) or

Ingenol 3-(4-chloro-2,5-dimethyl-pyrazole-3-carboxylate) or

Ingenol 3-(1,2,4-trimethylpyrrole-3-carboxylate) or

Ingenol 3-(1,3,5-trimethylpyrrole-2-carboxylate) or

Ingenol 3-(1-ethyl-3,5-dimethylpyrrole-2-carboxylate) or

Ingenol 3-(1-tert-butyloxycarbonyl-3,3-dimethylpyrrolidine-2-carboxylate) or

Ingenol 3-((25)-1-phenylpyrrolidine-2-carboxylate) or

Ingenol 3-(1-isopropyl-3,5-dimethyl-pyrazole-4-carboxylate) or

Ingenol 3-(5-ethyl-3-isopropyl-isoxazole-4-carboxylate) or

Ingenol 3-(2-methylindazole-3-carboxylate) or

Ingenol 3-(5-methyl-3-tert-butyl-isoxazole-4-carboxylate) or

Ingenol 3-(2-methyl-3-oxo-4-oxaspiro[4.5]dec-1-ene-1-carboxylate) or

Ingenol 3-(1-tert-butyl-3,5-dimethyl-pyrazole-4-carboxylate) or

Ingenol 3-(3,5-dimethylisothiazole-4-carboxylate) or

Ingenol 3-(5-iodo-3-methyl-isothiazole-4-carboxylate) or

Ingenol 3-(4-(4-methoxyphenyl)-2-methyl-pyrazole-3-carboxylate) or

Ingenol 3-(4-(2-methylphenyl)-2-methyl-pyrazole-3-carboxylate) or

Ingenol 3-(2-methyl-4-(4-methylsulfonylphenyl)pyrazole-3-carboxylate) or

Ingenol 3-(2-methyl-4-phenyl-pyrazole-3-carboxylate) or

Ingenol 3-(3,5-dimethyl-1-phenyl-pyrazole-4-carboxylate) or

Ingenol 3-(1,5-dimethyl-3-phenyl-pyrazole-4-carboxylate) or

Ingenol 3-(1-benzyl-3,5-dimethyl-pyrazole-4-carboxylate) or

Ingenol 3-(3,5-dimethyl-1-(tetrahydropyran-4-ylmethyppyrazole-4-carboxylate) or

Ingenol 3-(4-methyl-2-oxo-3H-thiazole-5-carboxylate) or

Ingenol 3-(2-methyl-4,5,6,7-tetrahydroindazole-3-carboxylate) or

Ingenol 3-(1,2-dimethylindole-3-carboxylate) or

Ingenol 3-(5-methoxy-1,2-dimethyl-indole-3-carboxylate) or

Ingenol 3-(1,3,5-trimethylpyrazole-4-carboxylate) or

Ingenol 3-(4-methyl-1,2,5-oxadiazole-3-carboxylate) or

Ingenol 3-(2-methoxy-4-methyl-thiazole-5-carboxylate) or

Ingenol 3-(4,5-dimethylisoxazole-3-carboxylate) or

Ingenol 3-(4-bromo-1-methyl-pyrazole-3-carboxylate) or

Ingenol 3-(1,3-dimethylindole-2-carboxylate) or

Ingenol 3-(5-methoxy-1,3-dimethyl-indole-2-carboxylate) or

Ingenol 3-(2,4-dimethyl-6-oxo-pyran-3-carboxylate) or

Ingenol 3-(1-methyl-3-phenyl-indole-2-carboxylate) or

Ingenol 3-(3-methyl-5-(trifluoromethypl)isoxazole-4-carboxylate) or

Ingenol 3-(1,3-dimethylpyrrole-2-carboxylate) or

Ingenol 3-(3,5-dimethyl-1-(2,2,2-trifluoroethyppyrazole-4-carboxylate) or

Ingenol 3-(1-cyclopropyl-2,5-dimethyl-pyrrole-3-carboxylate) or

Ingenol 3-(1,2,5-trimethylpyrrole-3-carboxylate) or

Ingenol 3-(2,4-dimethyl-1H-pyrrole-3-carboxylate) or

Ingenol 3-(1-methylpyrrole-2-carboxylate) or

Ingenol 3-(4-methyl-1H-pyrrole-2-carboxylate) or

Ingenol 3-(1,5-dimethylpyrrole-2-carboxylate) or

Ingenol 3-(3-methyl-1H-pyrrole-2-carboxylate) or

Ingenol 3-(1-cyclopropylpyrrole-2-carboxylate) or

Ingenol 3-(1-ethyl-2,4-dimethyl-pyrrole-3-carboxylate) or

Ingenol 3-(1-allyl-2,4-dimethyl-pyrrole-3-carboxylate) or

Ingenol 3-(1-(cyclopropylmethyl)-2,4-dimethyl-pyrrole-3-carboxylate) or

Ingenol 3-(1-(2-methoxyethyl)-2,4-dimethyl-pyrrole-3-carboxylate) or

Ingenol 3-(N-(3,5-dimethylisoxazol-4-yl)-N-methyl-carbamate) or

Ingenol 3-(N-(1,5-dimethylpyrazol-3-yl)-N-methyl-carbamate) or

Ingenol 3-(N-cyclopentyl-N-methyl-carbamate) or

Ingenol 3-(N-cyclopropyl-N-methyl-carbamate) or

Ingenol 3-(N-methyl-N-(2-pyridyl)-carbamate) or

Ingenol 3-(4-oxo-2,3-dihydroquinoline-1-carboxylate) or

Ingenol 3-(3,4-dihydro-2H-quinoline-1-carboxylate) or

Ingenol 3-(indoline-1-carboxylate) or

Ingenol 3-(azepane-1-carboxylate) or

Ingenol 3-(N-(4-chloro-phenyl)-N-methyl-carbamate) or

Ingenol 3-(N-(4-fluoro-phenyl)-N-methyl-carbamate) or

Ingenol 3-(N-methyl-N-(2-methoxy-phenyl)-carbamate) or

Ingenol 3-(N-methyl-N-(2-methyl-phenyl)-carbamate) or

Ingenol 3-(3-oxo-2,4-dyhidroquinoxaline-1-carboxylate) or

Ingenol 3-(N-ethyl-N-phenyl-carbamate) or

Ingenol 3-(2-trifluoromethyl-pyrrolidine-1-carboxylate) or

Ingenol 3-(3-azabicyclo[3.2.2]nonane-3-carboxylate) or

Ingenol 3-(2,3-dihydro-1,4-benzoxazine-4-carboxylate) or

Ingenol 3-(N-(2-fluoro-phenyl)-N-methyl-carbamate) or

Ingenol 3-(3-methyl-2,3-dihydro-1,4-benzoxazine-4-carboxylate) or

Ingenol 3-(2-trifluoromethyl-pyrrolidine-1-carboxylate) (ISOMER A) or

Ingenol 3-(2-trifluoromethyl-pyrrolidine-1-carboxylate) (ISOMER B) or

Ingenol 3-(N-methyl-N-(N-(tert-butyloxycarbonyl)-4-piperidyl)-carbamate) or

Ingenol 3-(N-methyl-N-(3-methyl-phenyl)-carbamate) or

Ingenol 3-(3,4-dihydro-2H-quinoxaline-1-carboxylate) or

Ingenol 3-(isoindoline-2-carboxylate) or

Ingenol 3-(N-methyl-N-(tetrahydropyran-4-ylmethyl)-carbamate) or

Ingenol 3-(N-methyl-N-(tetrahydropyran-4-yl)-carbamate) or

Ingenol 3-(N-methyl-N-(3-methoxy-phenyl)-carbamate) or

Ingenol 3-(N-cyclobutyl-N-methyl-carbamate) or

Ingenol 3-(N-allyl-N-methyl-carbamate) or

Ingenol 3-(N-methyl-N-prop-2-ynyl-carbamate) or

Ingenol 3-(N-methyl-N-(4-methylthiazol-2-yl)-carbamate) or

Ingenol 3-(N-(4-cyano-phenyl)-N-methyl-carbamate).

The term “storage stability” is intended to indicate that the composition exhibits chemical and physical stability characteristics that permit storage of the composition, at refrigeration or, preferably, room temperature for a sufficient period of time to make the composition commercially viable, such as at least 12 months, in particular at least 18 months, and preferably at least 2 years.

The term “chemical stability” or “chemically stable” is intended to indicate that no more than 10%, preferably no more than 6%, of the ingenol derivative degrades over the shelf-life of the product which is at least 12 months, preferably at least 18 months and more preferably 2 years. An approximation of chemical stability at room temperature is obtained by subjecting the composition to accelerated stability studies at 25° C. as described in Example 2 below. The term “physical stability” or “physically stable” is intended to mean that the composition retains its macroscopic and microscopic appearance over the shelf-life of the product, e.g. that the ingenol derivative does not precipitate from the solvent phase or that there is no visible phase separation of the solvent phase and the carrier phase.

The term “gel” is intended to mean a semisolid dosage form that contains one or more excipients, e.g. viscosity-increasing agents that provide stiffness to a solution or colloidal dispersion. The gel does not flow at low shear stress and exhibits plastic flow behaviour.

The term “solubilization capacity” is intended to indicate the ability of a solvent or mixture of solvents to dissolve a given substance, expressed as the amount required to effect complete solubilization of the substance.

The term “skin penetration” is intended to mean the diffusion of the active ingredient into the different layers of the skin, i.e. the stratum corneum, epidermis and dermis.

The term “skin permeation” is intended to mean the flux of the active ingredient through the skin into the systemic circulation or, in case of in vitro studies, the receptor fluid of the Franz cell apparatus used in the experiment.

The term “C₆₋₂₂ acylglyceride” is intended to indicate a triglyceride or a mixture of mono- and diglycerides or mono-, di- and triglycerides of C₆₋₂₂ fatty acids.

The term “medium chain triglycerides” is intended to indicate triglyceride esters of fatty acids with a chain length of 6-12 carbon atoms. A currently favoured example of medium chain triglycerides is a mixture of caprylic (C₈) and capric (C₁₀) triglycerides, e.g. available under the trade name Miglyol 812.

The term “substantially anhydrous” is intended to mean that the content of free water in the lipophilic carrier or vehicle is less than about 2% by weight, preferably less than about 1% by weight, such as less than 0.5% by weight, of the carrier or vehicle.

The term “acidic compound” is intended to indicate a compound capable of providing a net overall acidic environment in the composition and/or capable of neutralizing alkaline impurities detrimental to the chemical stability of the ingenol derivative.

The term “occlusive agent” is intended to indicate a lipid substance that forms a layer on the surface of the skin on application of the composition. The lipid layer forms a hydration barrier sufficient to result in reduction of transepidermal water loss, resulting in skin hydration.

### Embodiments

In an embodiment, the amount of the hydrophilic non-ionic surfactant in the solvent mixture is from about 5% by weight to about 90% by weight, or from about 10% by weight to about 70% by weight, in particular from about 30% by weight to about 60% by weight, such as from about 40% by weight to about 50% by weight of the mixture.

The hydrophilic surfactant may for instance be a polyethylene glycol ester of a vegetable oil containing at least 20 mole of ethylene oxide groups/mole of glyceride, such esters being selected from the group consisting of polyoxyethylene castor oil derivatives, e.g. PEG 20, 30, 35, 38, 40, 50 or 60 castor oil or PEG 20, 25, 30, 40, 45, 50, 60 or 80 hydrogenated castor oil, PEG 20 or 60 corn glycerides, PEG 20 or 60 almond glycerides or PEG 40 palm kernel oil, sodium laurate sulfate, a sucrose ester, e.g. sucrose stearate, sucrose distearate, sucrose cocoate or sucrose monolaurate, PEG cocoglyceride, PEG 8 caprylocaprate, polyglyceryl esters or linolenamide DEA.

In an embodiment, the amount of the lipophilic non-ionic co-surfactant in the solvent mixture is from about 5% by weight to about 90% by weight, or from about 10% by weight to about 50% by weight, in particular from about 20% by weight to about 40% by weight, such as from about 25% by weight to about 30% by weight of the mixture.

The lipophilic surfactant may be selected from the group consisting of monoglyceride esters of C₆₋₂₂ fatty acids such as glyceryl monocaprylate, glyceryl monocaprate, glyceryl monostearate, glyceryl monobehenate, diglyceride esters of C₆₋₂₂ fatty acids such as glyceryl dilaurate, mono- and diglyceride esters of C₆₋₂₂ fatty acids such as caprylic/capric mono- and diglycerides or glyceryl mono- and diricinoleate, propylene glycol esters of C₆₋₂₂ fatty esters such as propylene glycol monocaprylate or propylene glycol monolaurate, dialkylene glycol monoalkyl ethers such as diethylene glycol nnonoethyl ether, polyglyceryl C₆₋₂₂ fatty acid esters such as polyglyceryl-3-diisostearate, polyethylene glycol esters of a triglyceride/vegetable oil containing 4-8 mole of ethylene oxide groups/mole of glyceride such as PEG-6 corn oil, PEG-6 almond oil, PEG-6 apricot kernel oil, PEG-6 olive oil, PEG-6 peanut oil, PEG-6 palm kernel oil or hydrogenated palm kernel oil, PEG-6 triolein or PEG-8 corn oil, or polysorbates such as polysorbate 20, polysorbate 40, polysorbate 60 or polysorbate 80.

The ratio of surfactant:co-surfactant:oil may favourably be about 2:1:1 as this ratio may result in the formation of a monophasic system. Formation of a monophasic system is regarded as advantageous as such a system is generally physically stable, i.e. unlikely to result in phase separation.

It is known from the literature that including a large amount of surfactant(s) in a dermal composition is likely to cause significant skin irritation. The solvent mixture included in the present composition may be effective in itself to dissolve a sparingly soluble compound such as an ingenol derivative. The solubilisation capacity may make it possible to use a lower amount of surfactants and consequently decrease the risk of skin irritation while retaining a high biological activity. Thus, unlike the compositions for oral use disclosed in the publications discussed above, the solvent mixture only constitutes a minor proportion of the composition, the lipid carrier and optionally other excipients making up the remainder of the composition. Thus, the solvent mixture may constitute about 1-20% by weight, such as about 5-15% by weight or about 8-12% by weight or about 9-11% by weight, e.g. about 10% by weight, of the composition.

The oil included in the present composition may be selected from the group consisting of C₆₋₂₂ acylglycerides, volatile silicone oils, fatty alcohol esters of C₁₀₋₁₈ fatty acids, polyoxypropylene fatty alkyl ethers and pyrrolidones.

The C₆₋₂₂ acylglyceride may be a vegetable oil, e.g. sesame oil, sunflower oil, palm kernel oil, corn oil, safflower oil, olive oil, avocado oil, jojoba oil, grape kernel oil, canola oil, wheat germ oil, almond oil, cottonseed oil, peanut oil, walnut oil or soybean oil, a highly purified vegetable oil, e.g. medium chain triglycerides (caprylic/capric triglycerides), long chain triglycerides, castor oil, caprylic/capric mono- and diglycerides or caprylic/capric mono-, di- and triglycerides.

The fatty alcohol ester of C₁₀₋₁₈ fatty acids may be selected from, e.g., isopropyl myristate, isopropyl palmitate, isopropyl linoleate, isopropyl monooleate or isostearyl isostea rate.

The polyoxypropylene fatty alkyl ether may be selected from, e.g. polyoxypropylene-15-stearyl ether, polyoxypropylene-11-stearyl ether, polyoxypropylene-14-butyl ether, polyoxypropylene-10-cetyl ether or polyoxypropylene-3-myristyl ether.

The volatile silicone oil may be selected from, e.g. cyclomethicone or dimethicone,

The oil may be present in a concentration of about 0.5-10%, such as about 1-7%, about 2-5% or about 2-3%, such as about 2.5%, by weight of the composition.

In order to effect a highly efficient dissolution of the ingenol derivative it may be favourably to add a co-solvent to the composition. The co-solvent may be selected from the group consisting of lower alcohols, such as n-propanol, isopropanol, n-butanol, 2-butanol or benzyl alcohol, diols such as propylene glycol, a pyrrolidone such as N-methylpyrrolidone or N-hydroxyalkylpyrrolidone, an azone, ethyl acetate, dodecyl amine, diethylene glycol monoethyl ether, a fatty acid such as oleic or lauric acid, propylene carbonate, menthol or limolene. In particular, ingenol-3-angelate is soluble to freely soluble in the co-solvents benzyl alcohol (201-223 mg/ml), N-methylpyrrolidone (104-260 mg/ml), ethyl acetate (102-255 mg/ml), isopropanol (225-248 mg/ml) and diethylene glycol monoethyl ether (57-114 mg/ml). Some of these co-solvents, such as lower alcohols, may also, as required, act as penetration enhancers aiding the penetration of the ingenol derivative into the viable layers of the skin.

The lipid carrier may include an occlusive agent which may be selected from a mineral oil, e.g. liquid paraffin, or an isoparaffin such as isohexadecane.

To impart a desired viscosity to the present composition, it includes one or more lipophilic viscosity-increasing agents such as a paraffin composed of hydrocarbons with different chain lengths peaking at about C₄₀₋₄₄, e.g. petrolaturm or white soft paraffin, hard paraffin or a wax. The wax may be a mineral wax composed of a mixture of high molecular weight hydrocarbons, e.g. saturated C₃₅₋₇₀ alkanes, such as microcrystalline wax. Alternatively, the wax may be a silicone wax or a vegetable or animal wax, e.g. esters of C₁₄₋₃₂ fatty acids and C₁₄₋₃₂ fatty alcohols, such as beeswax or hydrogenated castor oil. Alternatively, the viscosity-increasing agent may be an inorganic substance such as fumed silica, e.g. available under the trade name Aerosil. The viscosity-increasing agent may also be selected from, e.g. magnesium stearate or polyethylene. The amount of viscosity-increasing agent may vary according to the viscosifying power of theagent, but may typically be in the range of about 0.5-40% by weight of the composition.

Ingenol-3-angelate is known to be a substance which is extremely sensitive to higher pH conditions (a pH above about 4.5 in aqueous compositions or in the presence of alkaline reacting substances in non-aqueous compositions) which contribute to the isomerization of the angelic acid ester to the tiglate ester. To ensure an adequate chemical stability of the substance throughout the shelf-life of the composition, it includes an acidic compound capable of neutralizing alkaline impurities which may be present in one or more of the excipients of the composition and which are detrimental to the chemical stability of ingenol-3-angelate. The acidic compound may favourably be selected from a low-molecular carboxylic acid, e.g. lactic acid, citric acid, salicylic acid or tartaric acid, which may be included in an amount of about 0.1-1% by weight of the composition. The acidic compound may also be selected from oleic acid and benzoic acid. There is a trend that carboxylic acids with a lower plc value exhibit a better stabilising effect, and it is expected that other suitable acidic compounds may be found among such carboxylic acids.

Examples of ingenol derivatives that may be included in the present composition are ingenol-3-angelate, ingenol-5-angelate, ingenol-20-angelate, 20-O-acetyl-ingenol-3-angelate and 20-deoxy-ingenol-3-angelate. A currently favoured ingenol derivative is ingenol-3-angelate, also known as ingenol-3-mebutate or PEP 005. The ingenol derivative may be included in the composition in an amount of about 0.001-0.5% by weight of the composition.

The composition of the invention may be used in the topical treatment of a dermal disease or condition. Examples of dermal diseases and conditions are actinic keratosis, seborrheic keratosis, skin cancer, such as basal cell carcinoma or squamous cell carcinoma, warts, keloids, scars, photoaged or photodamaged skin, or acne.

The term “skin cancer” is intended to include non-melanoma skin cancer, malignant melanoma, Merkel cell carcinoma, squamous cell carcinoma or basal cell carcinoma. Basal cell carcinomas include superficial basal cell carcinoma as well as nodular basal cell carcinoma.

The term “photodamaged skin” is intended to include cover fine lines, wrinkles and UV-ageing. UV ageing is often manifested by an increase in the epidermal thickness or epidermal atrophy and most notably by solar elastosis, the accumulation of elastin containing material just below the dermal-epidermal junction. Collagen and elastic fibres become fragmented and disorganised. At a cosmetic level this can be observed as a reddening and/or thickening of the skin resulting a a leathery appearance, skin fragility and irregular pigmentation, loss of tone and elasticity, as well as wrinkling, dryness, sunspots and deep furrow formation.

The term “warts” in the context of the present invention is intended to human papilloma virus (HPV) infections leading to formation of warts on the body, such as the skin, genitals and mouth.

The present composition may also be effective at reducing or minimizing scar tissue or improving cosmesis or functional outcome in a wound and scar reduction, wherein the wound is cutaneous, chronic or for example diabetes associated, and includes cuts and lacerations, surgical incisions, punctures, graces, scratches, compression wounds, abrasions, friction wounds, chronic wounds, ulcers, thermal effect wounds, chemical wounds, wounds resulting from pathogenic infections, skin graft/transplant donor and recipient sites, immune response conditions, oral wounds, stomach or intestinal wounds, damaged cartilage or bone, amputation sides and corneal lesions.

In a specific embodiment the composition comprises

0.001-0.5% by weight ingenol-3-angelate

2-3% by weight medium chain triglycerides

2-5% by weight caprylicicapric glycerides

4-6% by weight polyoxyl 40 hydrogenated castor oil

0.5-1% by weight benzyl alcohol

0.1-1% by weight carboxylic acid

0.5-40% by weight viscosity-increasing agent

40-95% by weight liquid paraffin

The invention is further described in the following examples which are not in any way intended to limit the scope of the invention as claimed.

### EXAMPLES

### Example 1

### Compositions Containing an Ingenol Derivative

Composition A

Ingenol-3-angelate 0.5 mg/g

Benzyl alcohol 9 mg/g

Miglyol 812 (medium chain triglycerides) 25 mg/g

Akoline MCM (caprylic/capric glycerides) 27 mg/g

Cremophor RH40 (polyoxyl 40 hydrogenated castor oil) 48 mg/g

Paraffin liquid 480.5-485.5 mg/g

White soft paraffin 400 mg/g

Hard paraffin 5-10 mg/g

Lactic acid S mg/g

Composition A1

Ingenol-3-angelate 0.5 mg/g

Benzyl alcohol 9 mg/g

Miglyol 812 (medium chain triglycerides) 25 mg/g

Akoline MCM (caprylic/capric glycerides) 27 mg/g

Cremophor RH40 (polyoxyl 40 hydrogenated castor oil) 48 mg/g

Paraffin liquid 890.5 mg/g

Hard paraffin 5-10 mg/g

Lactic acid 5 mg/g

Composition B

Ingenol-3-angelate 0.5 mg/g

Benzyl alcohol 9 mg/g

N-methylpyrrolidone 50 mg/g

Miglyol 812 25 mg/g

Akoline MCM 27 mg/g

Cremophor RH40 48 mg/g

Paraffin liquid 830.5 mg/g

Microcrystalline wax 50 mg/g

Lactic acid 10 mg/g

Composition B1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 880.5 mg/g

Lactic acid 10 mg/g

Composition C

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 788.5 mg/g

Microcrystalline wax 100 mg/g

Citric acid 2 mg/g

Composition C1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 888.5 mg/g

Citric acid 2 mg/g

Composition D

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 735.5 mg/g

Microcrystalline wax 150 mg/g

Citric acid 5 mg/g

Composition D1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 885.5 mg/g

Citric acid 5 mg/g

Composition E

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 485.5 mg/g

White soft paraffin 400 mg/g

Salicylic acid 5 mg/g

Composition E1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 885.5 mg/g

Salicylic acid 5 mg/g

Composition F

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 485.5 mg/g

White soft paraffin 400 mg/g

Tartaric acid 5 mg/g

Composition F1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin liquid 885.5 mg/g

Tartaric acid 5 mg/g

Composition G

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin, liquid 890.5 mg/g

Composition H

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin, liquid 855.5 mg/g

Oleic acid 5 mg/g

Composition H1

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin, liquid 885.5 mg/g

Oleic acid 5 mg/g

Composition I

Ingenol-3-angelate 0.5 mg/g

Benzyl alcohol 9 mg/g

Miglyol 812 25 mg/g

Akoline MCM 27 mg/g

Cremophor RH40 48 mg/g

Paraffin, liquid 880.5 mg/g

Oleic acid 10 mg/g

Composition J

PEP005 0.5 mg/g

Benzyl alcohol 9 mg/g

Miglyol 812 25 mg/g

Akoline MCM 27 mg/g

Cremophor RH40 48 mg/g

Paraffin, liquid 885.5 mg/g

Lactic acid 5 mg/g

Composition K

PEP005 0.5 mg/g

Benzyl alcohol 9 mg/g

Miglyol 812 25 mg/g

Akoline MCM 27 mg/g

Cremophor RH40 48 mg/g

Paraffin, liquid 880.5 mg/g

Lactic acid 10 mg/g

Composition L

PEP005 0.5 mg/g

Benzyl alcohol 9 mg/g

Miglyol 812 25 mg/g

Akoline MCM 27 mg/g

Cremophor RH40 48 mg/g

Paraffin, liquid 888.5 mg/g

Citric acid 2 mg/g

Composition M

PEP005 0.5 mg/g

Benzyl alcohol 9 mg/g

Miglyol 812 25 mg/g

Akoline MCM 27 mg/g

Cremophor RH40 48 mg/g

Paraffin, liquid 885.5 mg/g

Citric acid 5 mg/g

Composition N

PEP005 0.5 mg/g

Benzyl alcohol 9 mg/g

Miglyol 812 25 mg/g

Akoline MCM 27 mg/g

Cremophor RH40 48 mg/g

Paraffin, liquid 888.5 mg/g

Benzoic acid 2 mg/g

Composition O

PEP005 0.5 mg/g

Benzyl alcohol 9 mg/g

Miglyol 812 25 mg/g

Akoline MCM 27 mg/g

Cremophor RH40 48 mg/g

Paraffin, liquid 888.5 mg/g

Benzoic acid 5 mg/g

Composition P

PEP005 0.5 mg/g

Benzyl alcohol 9 mg/g

Miglyol 812 25 mg/g

Akoline MCM 27 mg/g

Cremophor RH40 48 mg/g

Paraffin, liquid 880.5 mg/g

Aerosil 200P 10 mg/g

Compositions A-P were prepared by mixing medium chain triglycerides, caprylic/capric glycerides and polyoxyl 40 hydrogenated castor oil and stirring the mixture for 15 min. at 50° C. with a magnetic stirrer. The requisite amount of acid was added to the mixture and stirred for 10 minutes after which the mixture was cooled to 30° C. The ingenol-3-angelate was dissolved in benzyl alcohol at room temperature and added to the mixture which was stirred to homogeneity by magnetic stirring. The resulting mixture was added to a mixture of liquid paraffin and viscosity-increasing agent and whisked at 30° C. until the resulting gel mixture was homogenous. The homogenized gel was cooled to room temperature and stored.

### Example 2

### Results of Stability Studies

50 ml clear glass bottles (Pyrex®) provided with blue screw caps protected with aluminium foil were used to store the compositions prepared in Example 1 for the purpose of stability testing. The bottles were placed in a heating cupboard at 25° C. The compositions were analysed for assay of ingenol-3-angelate and content of organic impurities by UPLC initially and after 2 and 4 weeks of storage (compositions G-P were analysed after 6 days and 4 weeks). Under these conditions, Compositions A-I all showed a promising stability profile in that the content of the main degradation products, 2-Butenoic acid, 2-methyl-, (1aR,2S,5R,5aS,65,8aS,9R,10aR)-4-(hydroxymethyl)-1a,2,5,5a,6,9,10,10a-octahydro-5a,6-dihydroxy-1,1,7,9-tetramethyl-11-oxo-1H-2,8a-methanocyclopenta[a]cyclopropa[e]cyclodecen-5-yl ester, (2Z)-(CAS Registry number: 91413-73-9), and 2-Butenoic acid, 2-methyl-, [(1aR,25,5R,5aS,6S,8aS,9R,10aR)-1a,2,5,5a,6,9,10,10a-octahydro-5,5a,6-trihydroxy-1,1,7,9-tetramethyl-11-oxo-1H-2,8a-methanocyclopenta[a]cyclopropa[e]cyclodecen-4-yl]methyl ester, (2Z)-(CAS Registry number: 83966-49-8), was less than 0.5%.

1. A substantially anhydrous pharmaceutical gel composition for cutaneous application comprising:
(a) an ingenol derivative in dissolved or solubilized form,
(b) a solvent mixture including
(i) a hydrophilic non-ionic surfactant,
(ii) a lipophilic non-ionic co-surfactant, and
(iii) an oil;
(c) an acidic compound;
(d) a co-solvent;
(e) a viscosity-increasing agent; and
(f) a pharmaceutically acceptable, substantially anhydrous, non-solvent lipid carrier.

2. A composition according to claim 1, wherein the ingenol derivative or analogue is selected from the group consisting of ingenol-3-angelate ingenol-5-angelate, ingenol-20-angelate, 20-O-acetyl-ingenol-3-angelate and 20-deoxy-ingenol-3-angelate.

3. A composition according to claim 2, wherein the ingenol derivative is ingenol-3-angelate.

4. A composition according to claim 1, wherein the amount of the hydrophilic non-ionic surfactant (b)(i) in the solvent mixture is from about 5% by weight to about 90% by weight, or from about 10% by weight to about 70% by weight, in particular from about 30% by weight to about 60% by weight, such as from about 40% by weight to about 50% by weight of the mixture.

5. A composition according to claim 4, wherein the hydrophilic non-ionic surfactant is a polyethylene glycol ester of a vegetable oil containing at least 20 mole of ethylene oxide groups/mole of glyceride, such esters being selected from athe group of esters consisting of polyoxyethylene castor oil derivatives, e.g. PEG 20, 30, 35, 38, 40, 50 or 60 castor oil or PEG 20, 25, 30, 40, 45, 50, 60 or 80 hydrogenated castor oil, PEG 20 or 60 corn glycerides, PEG 20 or 60 almond glycerides, PEG 40 palm kernel oil, sodium laurate sulfate, a sucrose ester, e.g. sucrose stearate, sucrose distearate, sucrose cocoate or sucrose monolaurate, PEG cocoglycerides, PEG 8 caprylocaprylate, polyglyceryl esters andef linolenamide DEA.

6. A composition according to claim 1, wherein the amount of the lipophilic non-ionic co-surfactant (b)(ii) in the solvent mixture is from about 5% by weight to about 90% by weight, or from about 10% by weight to about 50% by weight, in particular from about 20% by weight to about 40% by weight, such as from about 25% by weight to about 30% by weight of the mixture.

7. A composition according to claim 6, wherein the lipophilicnon-ionic co-surfactant (b)(ii) is selected from athe group of lipophilic surfactants consisting of monoglyceride esters of C₆₋₂₂ fatty acids such as glyceryl monocaprylate, glyceryl monocaprate, glyceryl monostearate, glyceryl monobehenate, diglyceride esters of C_{6- 22} fatty acids such as glyceryl dilaurate, mono- and diglyceride esters of C₆₋₂₂ fatty acids such as caprylic/capric mono- and diglycerides or glyceryl mono- and diricinoleate, propylene glycol esters of C₆₋₂₂ fatty esters such as propylene glycol monocaprylate or propylene glycol monolaurate, dialkylene glycol monoalkyl ethers such as diethylene glycol monoethyl ether, polyethylene glycol esters of a triglyceride/vegetable oil containing 4-8 mole of ethylene oxide groups/mole of glyceride such as PEG-6 corn oil, PEG-6 almond oil, PEG-6 apricot kernel oil, PEG-6 olive oil, PEG-6 peanut oil, PEG-6 palm kernel oil or hydrogenated palm kernel oil, PEG-6 triolein or PEG-8 corn oil and polysorbates such as polysorbate 20, polysorbate 40, polysorbate 60 or polysorbate 80.

8. A composition according to claim 1, wherein the acidic compound (c) is a low molecular carboxylic acid, e.g. lactic acid, citric acid, salicylic acid or tartaric acid.

9. A composition according to claim 1, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

10. A composition according to claim 1, wherein the oil (b)(iii) is selected from the group consisting of C₆₋₂₂ acylglycerides, volatile silicone oils, fatty alcohol esters of C₁₀₋₁₈ fatty acids, polyoxypropylene fatty alkyl ethers and pyrrolidones.

11. A composition according to claim 1, wherein the co-solvent (d) is selected from the group consisting of lower alcohols such as n-propanol, isopropanol, n-butanol or benzyl alcohol, diols such as propylene glycol, a pyrrolidone such as N-methylpyrrolidone or N-hydroxyalkylpyrrolidone, an azone, ethyl acetate, dodecyl amine, diethylene glycol monoethyl ether, a fatty acid such as oleic or lauric acid, propylene carbonate, menthol or limolene.

12. A composition according to claim 10, wherein the C₆₋₂₂ acylglyceride is selected from the group consisting of vegetable oils, e.g. sesame oil, palm kernel oil, corn oil, sunflower oil, safflower oil, olive oil, almond oil, avocado oil, jojoba oil, grape kernel oil, canola oil, wheat germ oil,almond oil, cottonseed oil, peanut oil, walnut oil or soybean oil, highly purified vegetable oils, e.g. medium chain triglycerides, long chain triglycerides or castor oil, and caprylic/capric mono-and diglycerides or caprylic/capric mono-, di-and triglycerides.

13. A composition according to claim 10, wherein the fatty alcohol ester of a C₁₀₋₁₈ fatty acid is isopropyl myristate, isopropyl palmitate, isopropyl linoleate, isopropyl monooleate or isostearyl isostearate.

14. A composition according to claim 10, wherein the polyoxypropylene fatty alkyl ether is polyoxypropylene-15-stearyl ether, polyoxypropylene-11-stearyl ether, polyoxypropylene-14-butyl ether, polyoxypropylene-10-cetyl ether or polyoxypropylene-3-myristyl ether.

15. A composition according to claim 10, wherein the volatile silicone oil is cyclomethicone or dimethicone.

16. A composition according to claim 10, wherein the pyrrolidone is N-alkylpyrrolidone, e.g. N-methylpyrrolidone, or N-hydroxyalkylpyrrolidone.

17. A composition according to claim 1, wherein the ratio of surfactant (b)(i):co-surfactant (b)(ii):oil (b)iii in the solvent mixture is 2:1:1.

18. A composition according to claim 1, wherein the solvent mixture (b) constitutes about 1-20% by weight, such as about 5-15% by weight or about 8-12% by weight or about 9-11% by weight, e.g. about 10% by weight, of the composition.

19. A composition according to claim 1, wherein the non-solvent lipid carrier (f) comprises a mineral oil, e.g. liquid paraffin, or an isoparaffin such as isohexadecane.

20. A composition according to claim 1, wherein the viscosity-increasing agent (e) is hard paraffin or a wax, e.g. hydrogenated castor oil, microcrystalline wax or silicone wax, a paraffin composed on hydrocarbons with different chain lengths peaking at about C₄₀₋₄₄, e.g. petrolatum or white soft paraffin, magnesium stearate, polyethylene or fumed silica.

21. A composition according to claim 1, wherein the composition further comprisesing a penetration enhancer selected from athe group of penetration enhancers consisting of menthol, nicotinamide, eucalyptol, or an azone.

22. A composition according to claim 1, wherein the composition further comprises from about 0.001%- to about 0.5% by weight of the ingenol derivative.

23. A method of treating a patient suffering from a dermal disease or condition, said method comprises
topically applying to the skin of the patient a composition according to claim 1 to the dermal disease or condition.

24. The composition of claim 23, wherein the dermal disease or condition is selected from a group of dermal diseases or conditions consisting of actinic keratosis, seborrheic keratosis, skin cancer, basal cell carcinoma, or squamous cell carcinoma, warts, keloids, scars, photoaged skin, photodamaged skin and acne.

25. A composition according to claim 2, wherein the amount of the hydrophilic surfactant (b)(i) in the solvent mixture is from about 5% by weight to about 90% by weight, or from about 10% by weight to about 70% by weight, in particular from about 30% by weight to about 60% by weight, such as from about 40% by weight to about 50% by weight of the mixture.

26. A composition according to claim 3, wherein the amount of the hydrophilic surfactant (b)(i) in the solvent mixture is from about 5% by weight to about 90% by weight, or from about 10% by weight to about 70% by weight, in particular from about 30% by weight to about 60% by weight, such as from about 40% by weight to about 50% by weight of the mixture.

27. A composition according to claim 25, wherein the hydrophilic surfactant (b)(i) is a polyethylene glycol ester of a vegetable oil containing at least 20 mole of ethylene oxide groups/mole of glyceride, such esters being selected from the group consisting of polyoxyethylene castor oil derivatives, e.g. PEG 20, 30, 35, 38, 40, 50 or 60 castor oil or PEG 20, 25, 30, 40, 45, 50, 60 or 80 hydrogenated castor oil, PEG 20 or 60 corn glycerides, PEG 20 or 60 almond glycerides, PEG 40 palm kernel oil, sodium laurate sulfate, a sucrose ester, e.g. sucrose stearate, sucrose distearate, sucrose cocoate or sucrose monolaurate, PEG cocoglycerides, PEG 8 caprylocaprylate, polyglyceryl esters or linolenamide DEA.

28. A composition according to claim 26, wherein the hydrophilic surfactant (b)(i) iis a polyethylene glycol ester of a vegetable oil containing at least 20 mole of ethylene oxide groups/mole of glyceride, such esters being selected from the group consisting of polyoxyethylene castor oil derivatives, e.g. PEG 20, 30, 35, 38, 40, 50 or 60 castor oil or PEG 20, 25, 30, 40, 45, 50, 60 or 80 hydrogenated castor oil, PEG 20 or 60 corn glycerides, PEG 20 or 60 almond glycerides, PEG 40 palm kernel oil, sodium laurate sulfate, a sucrose ester, e.g. sucrose stearate, sucrose distearate, sucrose cocoate or sucrose monolaurate, PEG cocoglycerides, PEG 8 caprylocaprylate, polyglyceryl esters or linolenamide DEA.

29. A composition according to claim 25, wherein the amount of the lipophilic non-ionic co-surfactant (b)(ii) in the solvent mixture is from about 5% by weight to about 90% by weight, or from about 10% by weight to about 50% by weight, in particular from about 20% by weight to about 40% by weight, such as from about 25% by weight to about 30% by weight of the mixture.

30. A composition according to claim , wherein the amount of the lipophilic non-ionic co-surfactant (b)(ii) in the solvent mixture is from about 5% by weight to about 90% by weight, or from about 10% by weight to about 50% by weight, in particular from about 20% by weight to about 40% by weight, such as from about 25% by weight to about 30% by weight of the mixture.

31. A composition according to claim 27, wherein the amount of the lipophilic non-ionic co-surfactant (b)(ii) in the solvent mixture is from about 5% by weight to about 90% by weight, or from about 10% by weight to about 50% by weight, in particular from about 20% by weight to about 40% by weight, such as from about 25% by weight to about 30% by weight of the mixture.

32. A composition according to claim 28, wherein the amount of the lipophilic non-ionic co-surfactant (b)(ii) in the solvent mixture is from about 5% by weight to about 90% by weight, or from about 10% by weight to about 50% by weight, in particular from about 20% by weight to about 40% by weight, such as from about 25% by weight to about 30% by weight of the mixture.

33. A composition according to claim 2, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

34. A composition according to claim 3, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

35. A composition according to claim 4, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

36. A composition according to claim 5, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

37. A composition according to claim 6, wherein the amount of oil (b)(iii) in the solvent mixture is from about 0.5% by weight to about 90% by weight, or from about 10% by weight to about 70% by weight, e.g. from about 15% by weight to about 40% by weight,such as from about 20% by weight to about 30% by weight of the mixture.

38. A composition according to claim 7, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

39. A composition according to claim 8, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

40. A composition according to claim 25, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

41. A composition according to claim 26, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

42. A composition according to claim 27, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

43. A composition according to claim 28, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

44. A composition according to claim 29, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

45. A composition according to claim 30, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

46. A composition according to claim 31, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

47. A composition according to claim 32, wherein the amount of oil (b)(iii) in the solvent mixture is from about 0.5% by weight to about 90% by weight, or from about 10% by weight to about 70% by weight, e.g. from about 15% by weight to about 40% by weight, such as from about 20% by weight to about 30% by weight of the mixture.

48. A composition according to claim 2, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

49. A composition according to claim 3, wherein the oil (b)(iii) selected from the group consisting of C6-22 acylglycerides, volatile silicone oils, fatty alcohol esters of C10-18 fatty acids, polyoxypropylene fatty alkyl ethers and pyrrolidones.

50. A composition according to claim 4, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

51. A composition according to claim 5, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

52. A composition according to claim 6, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

53. A composition according to claim 7, wherein the oil (b)(iii) is selected from the group consisting of C6-22 acylglycerides, volatile silicone oils, fatty alcohol esters of C 10-18 fatty acids, polyoxypropylene fatty alkyl ethers and pyrrolidones.

54. A composition according to claim 8, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

55. A composition according to claim 9, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

56. A composition according to claim 2, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

57. A composition according to claim 3, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

58. A composition according to claim 4, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

59. A composition according to claim 5, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

60. A composition according to claim 6, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

61. A composition according to claim 7, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

62. A composition according to claim 8, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

63. A composition according to claim 9, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

64. A composition according to claim 25, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

65. A composition according to claim 26, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

66. A composition according to claim 27, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

67. A composition according to claim 28, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

68. A composition according to claim 29, wherein the oil (b)(iii) is selected from the group consisting of C6-22 acylglycerides, volatile silicone oils, fatty alcohol esters of C 10-18 fatty acids, polyoxypropylene fatty alkyl ethers and pyrrolidones.

69. A composition according to claim 30, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

70. A composition according to claim 31, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

71. A composition according to claim 32, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

72. A composition according to claim 33, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

73. A composition according to claim 34, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

74. A composition according to claim 35, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

75. A composition according to claim 36, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

76. A composition according to claim 37, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

77. A composition according to claim 38, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

78. A composition according to claim 39, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

79. A composition according to claim 40, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

80. A composition according to claim 41, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

81. A composition according to claim 42, wherein the oil (b)(iii) is selected from the group consisting of C6-22 acylglycerides, volatile silicone oils, fatty alcohol esters of C 10-18 fatty acids, polyoxypropylene fatty alkyl ethers and pyrrolidones.

82. A composition according to claim 43, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

83. A composition according to claim 44, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

84. A composition according to claim 45, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

85. A composition according to claim 46, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

86. A composition according to claim 47, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

87. A composition according to claim 48, wherein the oil (b)(iii) is selected from the group consisting of C6-22 acylglycerides, volatile silicone oils, fatty alcohol esters of C10-18 fatty acids, polyoxypropylene fatty alkyl ethers and pyrrolidones.

88. A composition according to claim 2, wherein the ratio of surfactant (b)(i):co-surfactant (b)(ii):oil (b)(iii) in the solvent mixture is 2:1:1.

89. A composition according to claim 3, wherein the ratio of surfactant (b)(i):co-surfactant (b)(ii):oil (b)(iii) in the solvent mixture is 2:1:1.

90. A composition according to claim 4, wherein the ratio of surfactant (b)(i):co-surfactant (b)(ii):oil (b)(iii) in the solvent mixture is 2:1:1.

91. A composition according to claim 5, wherein the ratio of surfactant (b)(i):co-surfactant (b)(ii):oil (b)(iii) in the solvent mixture is 2:1:1.

92. A composition according to claim 6, wherein the ratio of surfactant b (i):co-surfactant (b)(ii):oil (b)(iii) in the solvent mixture is 2:1:1.

93. A composition according to claim 7, wherein the ratio of surfactant (b)(i):co-surfactant (b)(ii):oil (b)(iii) in the solvent mixture is 2:1:1.

94. A composition according to claim 8, wherein the ratio of surfactant (b)(i):co-surfactant (b)(ii):oil (b)(iii) in the solvent mixture is 2:1:1.

95. A composition according to claim 9, wherein the ratio of surfactant (b)(i):co-surfactant (b)(ii):oil (b)(iii) in the solvent mixture is 2:1:1.

96. A composition according to claim 10, wherein the ratio of surfactant (b)(i):co-surfactant (b)(ii):oil (b)(iii) in the solvent mixture is 2:1:1.

97. A composition according to claim 11, wherein the ratio of surfactant (b)(i):co-surfactant (b)(ii):oil (b)(iii) in the solvent mixture is 2:1:1.

98. A composition according to claim 12, wherein the ratio of surfactant (b)(i):co-surfactant (b)(ii):oil (b)(iii) in the solvent mixture is 2:1:1.

99. A composition according to claim 13, wherein the ratio of surfactant (b)(i):co-surfactant (b)(ii):oil (b)(iii) in the solvent mixture is 2:1:1.

100. A composition according to claim 14, wherein the ratio of surfactant (b)(i):co-surfactant (b)(ii):oil (b)(iii) in the solvent mixture is 2:1:1.

101. A composition according to claim 15, wherein the ratio of surfactant (b)(i):co-surfactant (b)(ii):oil (b)(iii) in the solvent mixture is 2:1:1.

102. A composition according to claim 16, wherein the ratio of surfactant (b)(i):co-surfactant (b)(ii):oil (b)(iii) in the solvent mixture is 2:1:1.

103. A composition according to claim 2, wherein the solvent mixture (b) constitutes about 1-20% by weight, such as about 5-15% by weight or about 8-12% by weight or about 9-11% by weight, e.g. about 10% by weight, of the composition.

104. A composition according to claim 3, wherein the solvent mixture (b) constitutes about 1-20% by weight, such as about 5-15% by weight or about 8-12% by weight or about 9-11% by weight, e g. about 10% by weight, of the composition.

105. A composition according to claim 4, wherein the solvent mixture (b) constitutes about 1-20% by weight, such as about 5-15% by weight or about 8-12% by weight or about 9-11% by weight, e.g. about 10% by weight, of the composition.

106. A composition according to claim 5, wherein the solvent mixture (b)constitutes about 1-20%, by weight, such as about 5-15% by weight or about 8-12% by weight or about 9-11% by weight, e.g. about 10% by weight, of the composition.

107. A composition according to claim 6, wherein the solvent mixture (b) constitutes about 1-20% by weight, such as about 5-15% by weight or about 8-12% by weight or about 9-11% by weight, e.g. about 10% by weight, of the composition.

108. A composition according to claim 7, wherein the solvent mixture (b) constitutes about 1-20% by weight, such as about 5-15% by weight or about 8-12% by weight or about 9-11% by weight, e.g. about 10% by weight, of the composition.

109. A composition according to claim 8, wherein the solvent mixture (b) constitutes about 1-20% by weight, such as about 5-15% by weight or about 8-12% by weight or about 9-11% by weight, e.g. about 10% by weight, of the composition.

110. A composition according to claim 9, wherein the solvent mixture (b) constitutes about 1-20% by weight, such as about 5-15% by weight or about 8-12% by weight or about 9-11% by weight, e.g. about 10% by weight, of the composition.

111. A composition according to claim 10, wherein the solvent mixture (b) constitutes about 1-20% by weight, such as about 5-15% by weight or about 8-12% by weight or about 9-11% by weight, e.g. about 10% by weight, of the composition.

112. A composition according to claim 11, wherein the solvent mixture (b) constitutes about 1-20% by weight, such as about 5-15% by weight or about 8-12% by weight or about 9-11% by weight, e.g. about 10% by weight, of the composition.

113. A composition according to claim 12, wherein the solvent mixture (1) constitutes about 1-20% by weight, such as about 5-15% by weight or about 8-12% by weight or about 9-11% by weight, e.g. about 10% by weight, of the composition.

114. A composition according to claim 13, wherein the solvent mixture (b) constitutes about 1-20% by weight, such as about 5-15% by weight or about 8-12% by weight or about 9-11% by weight, e.g. about 10% by weight, of the composition.

115. A composition according to claim 14, wherein the solvent mixture (b) constitutes about 1-20% by weight, such as about 5-15% by weight or about 8-12% by weight or about 9-11% by weight, e.g. about 10% by weight, of the composition.

116. A composition according to claim 15, wherein the solvent mixture (b) constitutes about 1-20% by weight, such as about 5-15% by weight or about 8-12% by weight or about 9-1 by weight, e.g. about 10% by weight, of the composition.

117. A composition according to claim 16, wherein the solvent mixture (b) constitutes about 1-20% by weight, such as about 5-15% by weight or about 8-12% by weight or about 9-11% by weight, e.g. about 10% by weight, of the composition.

118. A composition according to claim 17, wherein the solvent mixture (b) constitutes about 1-20% by weight, such as about 5-15% by weight or about 8-12% by weight or about 9-1 by weight, e.g. about 10% by weight, of the composition.

119. A composition according to claim 2, wherein the non-solvent lipid carrier (f) comprises a mineral oil, e.g. liquid paraffin, or an isoparaffin such as isohexadecane.

120. A composition according to claim 3, wherein the non-solvent lipid carrier (f) comprises a mineral oil, e.g. liquid paraffin, or an isoparaffin such as isohexadecane.

121. A composition according to claim 4, wherein the non-solvent lipid carrier (f) comprises a mineral oil, e.g. liquid paraffin, or an isoparaffin such as isohexadecane.

122. composition according to claim 5, wherein the non-solvent lipid carrier (f) comprises a mineral oil, e.g. liquid paraffin, or an isoparaffin such as isohexadecane.

123. A composition according to claim 6, wherein the non-solvent lipid carrier (f) comprises a mineral oil, e.g. liquid paraffin, or an isoparaffin such as isohexadecane.

124. A composition according to claim 7, wherein the non-solvent lipid carrier (f) comprises a mineral oil, e.g. liquid paraffin, or an isoparaffin such as isohexadecane.

125. A composition according to claim 8, wherein the non-solvent lipid carrier (f) comprises a mineral oil, e.g. liquid paraffin, or an isoparaffin such as isohexadecane.

126. A composition according to claim 9, wherein the non-solvent lipid carrier (f) comprises a mineral oil, e.g. liquid paraffin, or an isoparaffin such as isohexadecane.

127. A composition according to claim 10, wherein the non-solvent lipid carrier (f) comprises a mineral oil, e.g. liquid paraffin, or an isoparaffin such as isohexadecane.

128. A composition according to claim 11, wherein the non-solvent lipid carrier (f) comprises a mineral oil, e.g. liquid paraffin, or an isoparaffin such as isohexadecane.

129. A composition according to claim 12, wherein the non-solvent lipid carrier (f) comprises a mineral oil, e.g. liquid paraffin, or an isoparaffin such as isohexadecane.

130. A composition according to claim 13, wherein the non-solvent lipid carrier (f) comprises a mineral oil, e.g. liquid paraffin, or an isoparaffin such as isohexadecane.

131. A composition according to claim 14, wherein the non-solvent lipid carrier (f) comprises a mineral oil, e.g. liquid paraffin, or an isoparaffin such as isohexadecane.

132. A composition according to claim 15, wherein the non-solvent lipid carrier (f) comprises a mineral oil, e.g. liquid paraffin, or an isoparaffin such as isohexadecane.

133. A composition according to claim 16, wherein the non-solvent lipid carrier (f) comprises a mineral oil, e.g. liquid paraffin, or an isoparaffin such as isohexadecane.

134. A composition according to claim 17, wherein the non-solvent lipid carrier (f) comprises a mineral oil, e.g. liquid paraffin, or an isoparaffin such as isohexadecane.

135. A composition according to claim 18, wherein the non-solvent lipid carrier (f) comprises a mineral oil, e.g. liquid paraffin, or an isoparaffin such as isohexadecane.

136. A composition according to claim 2, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

137. A composition according to claim 3, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

138. A composition according to claim 4, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

139. A composition according to claim 5, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

140. A composition according to claim 6, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

141. A composition according to claim 7, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

142. A composition according to claim 8, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

143. A composition according to claim 9, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

144. A composition according to claim 10, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

145. A composition according to claim 11, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

146. A composition according to claim 12, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

147. A composition according to claim 13, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

141. A composition according to claim 14, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

148. A composition according to claim 15, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

149. A composition according to claim 16, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

150. A composition according to claim 17, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

151. A composition according to claim 18, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

152. A composition according to claim 19, wherein the viscosity-increasing agent (e) is hard paraffin or a wax, e.g. hydrogenated castor oil, microcrystalline wax or silicone wax, a paraffin composed on hydrocarbons with different chain lengths peaking at about C40-44, e.g. petrolatum or white soft paraffin, magnesium stearate, polyethylene or fumed silica.

153. A composition according to claim 2, wherein the composition further comprises a penetration enhancer selected from the group consisting of menthol, nicotinamide, eucal.yptol, or an azone.

154. A composition according to claim 3, wherein the composition further comprises a penetration enhancer selected from the group consisting of menthol, nicotinamide, eucalyptol, or an azone.

155. A composition according to claim 4, wherein the composition further comprises a penetration enhancer selected from the group consisting of menthol, nicotinamide, eucalyptol, or an azone.

156. A composition according to claim 5, wherein the composition further comprises a penetration enhancer selected from the group consisting of menthol, nicotinamide, eucalyptol, or an ozone.

157. A composition according to claim 6, wherein the composition further comprises a penetration enhancer selected from the group consisting of menthol, nicotinamide, eucalyptol, or an azone.

158. A composition according to claim 7, wherein the composition further comprises a penetration enhancer selected from the group consisting of menthol, nicotinamide, eucalyptol, or an azone.

159. A composition according to claim 8, wherein the composition further comprises a penetration enhancer selected from the group consisting of menthol, nicotinamide, eucalyptol, or an azone.

160. A composition according to claim 9, wherein the composition further comprises a penetration enhancer selected from the group consisting of menthol, nicotinamide, eucalyptol, or an azone.

161. A composition according to claim 10, wherein the composition further comprises a penetration enhancer selected from the group consisting of menthol, nicotinamide, eucalyptol, or an azone.

162. A composition according to claim 11, wherein the composition further comprises a penetration enhancer selected from the group consisting of menthol, nicotinamide, eucalyptol, or an azone.

163. A composition according to claim 12, wherein the composition further comprises a penetration enhancer selected from the group consisting of menthol, nicotinamide, eucalyptol, or an azone.

164. A composition according to claim 13, wherein the composition further comprises a penetration enhancer selected from the group consisting of menthol, nicotinamide, eucalyptol, or an azone.

165. A composition according to claim 14, wherein the composition further comprises a penetration enhancer selected from the group consisting of menthol, nicotinamide, eticalyptol, or an azone.

166. A composition according to claim 15, wherein the composition further comprises a penetration enhancer selected from the group consisting of menthol, nicotinamide, eucalyptol, or an azone.

167. A composition according to claim 16, wherein the composition further comprises a penetration enhancer selected from the group consisting of menthol, nicotinamide, eucalyptol, or an azone.

168. A composition according to claim 17, wherein the composition further comprises a penetration enhancer selected from the group consisting of menthol, nicotinamide, eucalyptol, or an azone.

169. A composition according to claim 18, wherein the composition further comprises a penetration enhancer selected from the group consisting of menthol, nicotinamide, eucalyptol, or an azone.

170. A composition according to claim 19, wherein the composition further comprises a penetration enhancer selected from the group consisting of menthol, nicotinamide, eucalyptol, or an azone.

171. A composition according to claim 20, wherein the composition further comprises a penetration enhancer selected from the group consisting of menthol, nicotinamide, eucalyptol, or an azone.

172. A composition according to claim 2, wherein the composition further comprises from about 0.001 to about 0.5% by weight of the ingenol derivative.

173. A composition according to claim 3, wherein the composition further comprises from about 0.001 to about 0.5% by weight of the ingenol derivative.

174. A composition according to claim 4, wherein the composition further comprises from about 0.001 to about 0.5% by weight of the ingenol derivative.

175. A composition according to claim 5, wherein the composition further comprises from about 0.001 to about 0.5% by weight of the ingenol derivative.

176. A composition according to claim 6, wherein the composition further comprises from about 0.001 to about 0.5% by weight of the ingenol derivative.

177. A composition according to claim 7, wherein the composition further comprises from about 0.001 to about 0.5% by weight of the ingenol derivative.

178. A composition according to claim 8, wherein the composition further comprises from about 0.001 to about 0.5% by weight of the ingenol derivative.

179. A composition according to claim 9, wherein the composition further comprises from about 0.001 to about 0.5% by weight of the ingenol derivative.

180. A composition according to claim 10, wherein the composition further comprises from about 0.001 to about 0.5% by weight of the ingenol derivative.

181. A composition according to claim 11, wherein the composition further comprises from about 0.001 to about 0.5% by weight of the ingenol derivative.

182. A composition according to claim 12, wherein the composition further comprises from about 0.001 to about 0.5% by weight of the ingenol derivative.

183. A composition according to claim 13, wherein the composition further comprises from about 0.001 to about 0.5% by weight of the ingenol derivative.

184. A composition according to claim 14, wherein the composition further comprises from about 0.001 to about 0.5% by weight of the ingenol derivative.

185. A composition according to claim 15, wherein the composition further comprises from about 0.001 to about 0.5% by weight of the ingenol derivative.

186. A composition according to claim 16, wherein the composition further comprises from about 0.001 to about 0.5% by weight of the ingenol derivative.

187. A composition according to claim 17, wherein the composition further comprises from about 0.001 to about 0.5% by weight of the ingenol derivative.

188. A composition according to claim 18, wherein the composition further comprises from about 0.001 to about 0.5% by weight of the ingenol derivative.

189. A composition according to claim 19, wherein the composition further comprises from about 0.001 to about 0.5% by weight of the ingenol derivative.

190. A composition according to claim 20, wherein the composition further comprises from about 0.001 to about 0.5% by weight of the ingenol derivative.

191. A composition according to claim 21, wherein the composition further comprises from about 0.001 to about 0.5% by weight of the ingenol derivative.
